(19) United States
(12) Patent Application Publication
(10) Pub. No.: US 2014/0275440 A1
(43) Pub. Date: Sep. 18, 2014

(54) DIAMINE MONOMER HAVING SIDE CHAIN, POLYIMIDE COMPOUND HAVING SIDE CHAIN AND MANUFACTURING METHOD THEREOF

(71) Applicant: UNIMICRON TECHNOLOGY CORP., Taoyuan Hsien (TW)

(72) Inventor: HAN-PEI HUANG, TAOYUAN COUNTY (TW)

(73) Assignee: UNIMICRON TECHNOLOGY CORP., TAOYUAN HSIEN (TW)

(21) Appl. No.: 13/845,279

(22) Filed: Mar. 18, 2013

(51) Int. Cl. C08G 73/10; C07C 213/00; C07C 217/90

(52) U.S. Cl. CPC C08G 73/1071; C07C 217/90; C07C 213/00
USPC 525/436; 564/430; 564/428; 564/427

(57) ABSTRACT

A diamine monomer having a large side chain R is provided. The large side chain would interrupt the symmetry and regularity of diamine monomer. The diamine monomer has the general formula shown as formula (V) below:

[chemical structure]

The functional group R includes α-substitution cycloalkene with at least a tertiary carbon atom, cycloalkene with at least a tertiary carbon atom, cycloalkane with at least a tertiary carbon atom, α-substitution phenyl, phenyl, α-substitution naphthalyl, naphthalyl, α-substitution phenanthrenyl, phenanthrenyl, α-substitution anthracenyl, anthracenyl, α-substitution adamantyl, adamantyl, α-substitution diamantyl and diamantyl.

### BACKGROUND

1. Technical Field

The invention relates to diamine monomer, polyimide compound and the manufacturing method thereof, and in particular, to diamine monomer with side chain, polyimide compound with side chain and the manufacturing method thereof.

2. Description of Related Art

The intermolecular forces of polyimide are strong, which reflects on its bond strength and thermal resistance. Therefore, polyimide is usually used as an insulating material or a heat-resisting material, and is widely implemented in the electrical field and the photoelectrical field. Nevertheless, the strong intermolecular forces of polyimide make it hard to dissolve in various kinds of the organic solvents, and thus the machinability of polyimide decreases. So far, several experimental reports about modifying the property of polyimide are published, and those researchers intended to increase the solubility of polyimide while the thermal property can be maintained at the same time.

### SUMMARY

The present invention provides diamine monomer having a side chain.

The present invention provides a manufacturing method of diamine monomer.

The present invention provides polyimide compound having a side chain.

The present invention provides a manufacturing method of polyimide compound.

The present invention provides diamine monomer having the side chain. The side chain of diamine monomer can interrupts the symmetry of diamine monomer in structure, and the regularity of diamine monomer in molecular arrangement. The above-mentioned side chain is selected from the group consisting of: α-substitution cycloalkene having at least a tertiary carbon atom, cycloalkane having at least a tertiary carbon atom, cycloalkene having at least a tertiary carbon atom, phenyl, α-substitution phenyl, naphthalyl, α-substitution naphthalyl, anthracenyl, α-substitution anthracenyl, phenanthrenyl, α-substitution phenanthrenyl, aromatic hydrocarbons, α-substitution aromatic hydrocarbons, adamantyl, α-substitution adamantyl, diamantyl, and α-substitution diamantyl.

The present invention provides a manufacturing method of diamine monomer. The method comprising: (A) The solution containing the bromo compound, the hydroquinone compound, and the benzene is heating refluxed for distillation. The solid hydroquinone compound is separated out in the distillation. (B) The solution containing hydroquinone compound, potassium carbonate (K₂CO₃), 4-nitrochlorobenzene, and dimethylformamide (DMF) is heated. The bis(4-nitrophenoxy) compound precipitates out from the distilled water (C) The solution containing bis(4-nitrophenoxy) compound, hydrazine monohydrate, ethanol, and Pd/C catalyst is heating refluxed to separate solid diamine monomer.

The present invention provides polyimide compound having the side chain. The polyimide compound is made of the above-mention diamine monomer. The side chain of the polyimide compound interrupts the symmetry of polyimide compound in structure, and the regularity of polyimide compound in molecular arrangement. The above-mentioned side chain is selected from the group consisting of: α-substitution cycloalkene having at least a tertiary carbon atom, a cycloalkane having at least a tertiary carbon atom, cycloalkene having at least a tertiary carbon atom, phenyl, α-substitution phenyl, naphthalyl, α-substitution naphthalyl, anthracenyl, α-substitution anthracenyl, phenanthrenyl, α-substitution phenanthrenyl, α-substitution aromatic hydrocarbons, aromatic hydrocarbons, adamantyl, α-substitution adamantyl, diamantyl, α-substitution diamantyl.

The present invention provides a manufacturing method of polyimide compound. The method comprising: (A) Under anhydrous condition, dianhydride monomer and diamine monomer are dissolved into cresol by stirring to from polyamic acid compound solution. (B) The 5-10 drops of isoquinoline are added to the polyamic acid compound solution. The solution containing polyamic acid compound solution and isoquinoline is heating refluxed to form polyimide compound solution. (C) The polyimide compound solution is cooled and added to the ethanol. The solid polyimide compound then precipitates out.

To sum up, the present invention provides diamine monomer with the side chain, polyimide compound having the side chain, and the manufacturing method thereof. The above-mention diamine monomer having large side chain has the property of interrupted symmetry in structure and poor regularity in molecular arrangement. Thus, the intermolecular force of polyimide compound is attenuated and the solubility of polyimide compound increases.

In order to further appreciate the characteristic and technical contents of the present invention, references are hereunder made to the detailed descriptions and appended drawings in connection with the present invention. However, the appended drawings are merely shown for exemplary purpose rather than being used to restrict the scope of the present invention.

### DESCRIPTION OF THE EXEMPLARY EMBODIMENTS

The present invention provides diamine monomer (d) with side chain. The general formula of diamine monomer (d) is shown as formula (I):

[chemical structure]

The functional group R is selected from the larger functional group consisting of: α-substitution cycloalkene with at least a tertiary carbon atom, cycloalkane with at least a tertiary carbon atom, cycloalkene with at least a tertiary carbon atom, α-substitution phenyl, phenyl, α-substitution naphthalyl, naphthalyl, α-substitution anthracenyl, anthracenyl, α-substitution phenanthrenyl, phenanthrenyl, α-substitution aromatic hydrocarbons, aromatic hydrocarbons, adamantyl, α-substitution adamantyl, diamantyl, α-substitution diamantyl.

Next, the manufacturing method of above-mention diamine monomer (d) is introduced. The manufacturing method comprising three main steps: forming hydroquinone compound (b), forming bis(4-nitrophenoxy) compound (c), and forming diamine monomer (d). It is worth noting that the “R” mentioned hereafter is one of the larger functional groups listed above and the detailed description of the larger functional groups would be omitted thereafter.

The formation of hydroquinone compound (b) comprises the following steps. Firstly, bromo compound (a), hydroquinone compound, and benzene are added into the reactor in nitrogen ambient. Next, the reactor is heating refluxed for 3-4 days. The reactor might be the three-necked flask. It is worth noting that nitrogen ambient might prevent the reaction from the influence of the air and the moisture. In addition, the reaction temperature of the process of the heating reflux is about 80-85° C. as the temperature benzene boiling point. After the reaction finished, solid hydroquinone compound (b) would be separated out. Solid hydroquinone compound (b) might be obtained directly by filtration. The general formula of bromo compound (a) is shown as formula (II) below:

[chemical structure]

The general formula of hydroquinone compound (b) is shown as formula (III) below:

[chemical structure]

Continued from the preceding paragraph, hydroquinone compound (b) obtained from filtration is washed by heated water to wash away the excess hydroquinone compound and hydrogen bromide. Hydrogen bromide is the byproduct of the reaction. The temperature of the heated water is about 50-70° C. Next, wet hydroquinone compound (b) is vacuum dried to remove the solvent and the moisture and to obtain solid hydroquinone compound (b). Then, solid hydroquinone compound (b) is recrystallized to enhance the purity of hydroquinone compound (b). The reaction of forming hydroquinone compound (b) is listed as reaction (I) below:

[chemical structure]

The formation of bis(4-nitrophenoxy) compound (c) comprises the following steps. Firstly, purified hydroquinone compound (b), potassium carbonate, 4-halogen-nitrobenzene, and dimethylformamide are added to the reactor under nitrogen ambient and heated to form bis(4-nitrophenoxy) compound (c). In the present embodiment, 4-halogen-nitrobenzene is 4-chloronitrobenzene. Alternatively, 4-halogen-nitrobenzene might also be 4-fluoronitrobenzene in other embodiment. The reaction period is 8-14 hours and the reaction temperature is about 120-140° C. After this reaction, solid bis(4-nitrophenoxy) compound (c) is precipitated out by adding distilled water.

Continued from the preceding paragraph, next, bis(4-nitrophenoxy) compound (c) is washed by the distilled water to wash away the excess solvent and the remaining residues. Next, solid bis(4-nitrophenoxy) compound (c) is obtained by vacuum dry. Then, solid bis(4-nitrophenoxy) compound (c) is dissolved to recrystallize, so as to enhance the purity of bis(4-nitrophenoxy) compound (c). The general formula of bis(4-nitrophenoxy) compound (c) is shown as formula (IV) below:

[chemical structure]

The reaction of forming bis(4-nitrophenoxy) compound (c) is listed as reaction (II) below:

[chemical structure]

The formation of diamine monomer (d) comprises the following steps. Firstly, bis(4-nitrophenoxy) compound (c), hydrazine monohydrate, ethanol, and Pd/C catalyst are added to the reactor and are heating refluxed for 1-2 days. The temperature of the heating reflux process is about 95-115° C. as the temperature of the boiling point of the ethanol. Next, diamine monomer (d) is washed by heated distilled water. The temperature of the heated water is about 70-80° C. Then, diamine monomer (d) is vacuum dried. Then, diamine monomer (d) is recrystallized to enhance the purity of diamine monomer (d). The general formula of diamine monomer (d) is shown as formula (I) below:

[chemical structure]

The reaction of forming diamine monomer (d) is listed as reaction (III) below:

[chemical structure]

In the following paragraph, adamantyl is chosen to be the functional group R of diamine monomer (d) in this embodiment. However, the embodiment is merely shown for exemplary purpose rather than being used to restrict the scope of the present invention.

### First Embodiment

The diamine monomer (d) in the first embodiment of the present invention is 1,4-bis(4-aminophenoxy)-2-adamantyl benzene, the formula of the 1,4-bis(4-aminophenoxy)-2-adamantyl benzene is shown as formula (V) below

[chemical structure]

The formation of the diamine monomer comprises the following three steps: forming the 2-adamantyl hydroquinone, forming 1,4-bis(4-notrophenoxy)-2-adamantyl benzene, and forming the 1,4-bis(4-aminophenoxy)-2-adamantyl benzene.

The formation of the 2-adamantyl hydroquinone comprises the following steps. Firstly, 15 g of 1-bromoadamantane (69.77 mmol), 15.35 g of hydroquinone compound (139.5 mmol), and 75 ml of benzene are added into the 250 ml of the three-necked flask under nitrogen ambient. The solution is heating refluxed for 72 hours. The temperature of the reaction is about 80-85° C. as the boiling point of benzene. It is worth noting that a large amount of white fumes is produced in the process of reaction and the color of the solution would become deeper.

After the reaction, solid 2-adamantyl hydroquinone is separated out and is obtained directly by the method of filtration. After the filtration, solid 2-adamantyl hydroquinone is washed by the heated water to remove the remaining hydroquinone compound. Next, 2-adamantyl hydroquinone is vacuum dried and recrystallized by toluene to enhance the purity of the 2-adamantylhydroquinone. Then, 12.21 g of 2-adamantylhydroquinone is obtained. The appearance of 2-adamantylhydroquinone is yellow transparent, needle-like crystal, the yield of 2-adamantylhydroquinone is 71.73%, the melting point (mp) of 2-adamantylhydroquinone is 217-219° C., and the property of 2-adamantylhydroquinone is listed as below:

IR (KBr) 3437, 3390, 3047, 3015, 2898, 2848, 1597, 1507, 1457 cm−1; MS (EI) m/z 244 (M+, 100), 186 (12).

Formation of 1,4-bis(4-notrophenoxy)-2-adamantyl benzene comprises the following steps. Firstly, 2 g of 2-adamantyl hydroquinone (8.197 mmol), 2.7 g of 4-nitrochlorobenzene (17.14 mmol), 1.5 g of potassium carbonate (10.87 mmol), and 40 ml of dimethylformamide are added to the 125 ml three-necked flask under the nitrogen ambient. The solution is heating refluxed at 130-135° C. for 12 hours.

After the reaction, the color of the solution would be dark brown. Then, the pale yellow solid 1,4-bis(4-notrophenoxy)-2-adamantyl benzene is precipitated out by adding the distilled water. Then, 1,4-bis(4-notrophenoxy)-2-adamantyl benzene is washed several times by the distilled water and vacuum dried. Next, solid 1,4-bis(4-notrophenoxy)-2-adamantyl benzene is recrystallized to enhance the purity. Then, 3.51 g of pale yellow fibrous crystal 1,4-bis(4-notrophenoxy)-2-adamantyl benzene is obtained with 88.1% of the yield, and approximately 190-192° C. of the melting point. The property of 1,4-bis(4-notrophenoxy)-2-adamantyl benzene is listed as below:

IR (KBr) 3078, 2903, 2847, 1608, 1591, 1512, 1478, 1341 cm−1; MS (EI) m/z 486 (M+, 100), 152 (40), 78 (70).

Formation of 1,4-bis(4-aminophenoxy)-2-adamantyl benzene comprises the following steps. Firstly, 2 g of 1,4-bis(4-notrophenoxy)-2-adamantyl benzene (4.12 mmol), 20 ml of hydrazine monohydrate, 0.09 g of Pd/C catalyst (10%), and 70 ml of ethanol are added into the 250 ml of three-necked flask under nitrogen ambient. Then, the solution is heating refluxed for 24 hours at 110° C. Next, the solution after heating reflux is filtered directly and is condensed.

After cooling down, pale yellow solid 1,4-bis(4-aminophenoxy)-2-adamantyl benzene is precipitated out. Then, 1,4-bis(4-aminophenoxy)-2-adamantyl benzene is washed several times with the distilled water and vacuum dried. Next, solid 1,4-bis(4-aminophenoxy)-2-adamantyl benzene is recrystallized to enhance the purity. The 1.54 g of pale brown crystal 1,4-bis(4-aminophenoxy)-2-adamantyl benzene is obtained with 87.85% of the yield, and approximately 171 to 172° C. of the melting point. The property of 1,4-bis(4-aminophenoxy)-2-adamantyl benzene is listed as below:

IR (KBr) 3469, 3406, 3383, 3336, 3215, 3055, 2902, 2848, 1626, 1505, 1479, 1451, 1402 cm−1; MS (EI) m/z 426 (M+, 100), 213 (18), 107 (54).

The above-mentioned is about the diamine monomer (d) provided in present invention. The polyimide compound (g) made of the above-mentioned diamine monomer (d) is introduced in the following paragraph. The general formula of polyimide compound (g) is shown as formula (VI) below:

[chemical structure]

R′ is selected from the group consisting of:

[chemical structure]

According to the above-mentioned formula of polyimide compound (g), polyimide compound (g) in the present invention has a large side chain. The large side chain would interrupt the symmetry of polyimide compound in structure and the regularity of polyimide compound in molecular arrangement.

Next, the manufacturing method of the polyimide compound (g) is introduced. The manufacturing method of polyimide compound (g) comprises the method of thermal imidization and chemical imidization. It is worth noting that, the “R′” mentioned hereafter is one of the above-mentioned eight functional groups cited as I to VIII and the detailed description of the eight functional groups would be omitted thereafter. In addition, polyimide compound made by thermal imidization would be marked as (g′), and polyimide compound made by chemical imidization would be marked as (g″), so as to discriminate polyimide compound made by the method of thermal imidization from which made by the method of chemical imidization.

The method of thermal imidization to manufacture the polyimide compound (g′) would be introduced firstly. The method of thermal imidization comprises two main steps: forming polyamic acid compound (f) and forming polyimide compound (g′).

Formation of polyamic acid compound (f) comprises the following steps. Firstly, diamine monomer (d) is dissolved in anhydrous N-methyl-2-pyrrolidone. The solution is stirred in an ice bath at 0-4° C. Then, dianhydride monomer (e) with same mole as the diamine monomer (d) is added with stirring for 1-2 hours. After that, the solution containing diamine monomer (d) and dianhydride monomer (e) is stirred in the room temperature for 4-5 hours to from polyamic acid compound (f). The general formula of the dianhydride monomer (e) is shown as formula (VII) below:

[chemical structure]

The general formula of the polyamic acid compound (f) is shown as formula (VIII) below:

[chemical structure]

Formation of solid polyimide compound (g) comprises the following steps. The polyamic acid compound (f) is coated on the glass plate to form the film. Then, the glass plate is backed under vacuum for 100-300° C. Then, polyamic acid compound (f) is cyclized to form the solid polyimide compound (g) under the high temperature. The temperature of reaction is about 300-350° C. The general formula of the solid polyimide compound (g) is shown as formula (IX) below:

[chemical structure]

The method of chemical imidization to manufacture the polyimide compound (g″) is introduced next. The method of chemical imidization comprises three main steps: forming polyamic acid compound (f), forming liquid polyimide compound (g″) and forming solid polyimide compound (g″).

Formation of polyamic acid compound (f) comprises the following steps. Dianhydride monomer (e) and diamine monomer (d) are dissolved into cresol with stirred under anhydrous condition to form polyamic acid compound (f). The temperature of the reaction is 20-30° C., and the time of the reaction is 6-7 hours.

Next, formation of liquid polyimide compound (g″) comprises the following steps. The 5-10 drops of isoquinoline are dropped into above-mentioned polyamic acid compound (f). The solution is heating refluxed at 100-200° C. for 8-12 hours, to form liquid polyimide compound (h). Herein, isoquinoline is used as dehydrating agent.

It is worth noting that, the step of forming polyimide compound (g″) is a dehydrating step, and thus the process must be conducted under anhydrous condition. In general, nitrogen or other inert gas would be introduced to avoid the influence of the moisture. In addition, cresol could be p-cresol or m-cresol, and the present invention is not limited thereto. Furthermore, the heating reflux process of solution contained polyamic acid compound (f) and isoquinoline could be divided into three steps. Firstly, the solution is heating refluxed at 90-100° C. for 2-4 hours. After that, the temperature of the reaction would be raised to 140-150° C. and the solution is heating refluxed for 2-4 hours. Next, the reaction temperature would be raised to 200-210° C. and the solution is heating refluxed for 2-4 hours.

Continued from the preceding paragraph, the above-mentioned solution of polyimide compound (g″) is cooled to 20-30° C., and added into ethanol. Then, solid polyimide compound (g″) is precipitated out. The solid polyimide compound (g″) could be distilled by ethanol, and be obtained by the method of extraction to enhance the purity. The reaction temperature of the distillation is about 70-80° C. as the boiling point of the ethanol. The reaction time is 4-6 hours. Then, the solid polyimide compound (g″) is obtained by the process of vacuum dried.

In order to further appreciate the contents of the present invention, the diamine monomer (d) obtained from the first embodiment hereunder is reacted with the above-mentioned dianhydride monomers (eI-eVIII) individually to from polyimide compounds (gI-gVIII) as the second to ninth embodiments. It is worth noting that dianhydride monomers mentioned above have eight different R′ groups marked as I-VIII, thus different dianhydride monomers (e) would be marked as eI to eVIII, so as to distinguish them. That is to say, the dianhydride monomer (e) with the I group would be marked as eI. Besides, polyamic acid compounds (f) and polyimide compounds (g) made by dianhydride monomer (eI-eVIII) would be marked as fI-fVIII and gI-gVIII to distinguish them. However, the present invention is not limited thereto.

On the other hand, in the present invention, both of the thermal imidization and the chemical imidization are utilized to form the above-mentioned eight polyimide compounds, so as to make the comparison. In other words, all the eight embodiments listed below comprises both the polyimide compound (g′) and polyimide compound (g″).

### Second Embodiment

1,4-bis(4-aminophenoxy)-2-adamantyl benzene is taken as the diamine monomer (d) of the polyimide (gI) in the second embodiment of the present invention. The group I is taken as the main chain of the dianhydride monomer (eI) of the polyimide (gI) in the second embodiment of the present invention. The formula of the polyimide (gI) in the second embodiment of present invention is shown as formula (X) below:

[chemical structure]

It is worth noting that the formation of the polyimide (gI) comprises thermal imidization to form polyimide (g′I) and chemical imidization to form polyimide (g″I). The method of thermal imidization and chemical imidization had already been described above and would be omitted thereafter.

### Third Embodiment

1,4-bis(4-aminophenoxy)-2-adamantyl benzene is taken as the diamine monomer (d) of the polyimide (gII) in the third embodiment of the present invention. The group II is taken as the main chain of the dianhydride monomer (eII) of the polyimide (gII) in the third embodiment of the present invention. The formula of the polyimide (gII) in third embodiment of present invention is shown as formula (XI) below:

[chemical structure]

It is worth noting that the formation of the polyimide (gII) comprises thermal imidization to form polyimide (g′II) and chemical imidization to form polyimide (g″II). The method of thermal imidization and chemical imidization had already been described above and would be omitted thereafter.

### Fourth Embodiment

1,4-bis(4-aminophenoxy)-2-adamantyl benzene is taken as the diamine monomer (d) of the polyimide (gIII) in the fourth embodiment of the present invention. The group III is taken as the main chain of the dianhydride monomer (eIII) of the polyimide (gIII) in the fourth embodiment of the present invention. The formula of the polyimide (gIII) in the fourth embodiment of the present invention is shown as formula (XII) below:

[chemical structure]

It is worth noting that the formation of the polyimide (gIII) comprises thermal imidization to form polyimide (g′III) and chemical imidization to form polyimide (g″III). The method of thermal imidization and chemical imidization had already been described above and would be omitted thereafter.

### Fifth Embodiment

1,4-bis(4-aminophenoxy)-2-adamantyl benzene is taken as the diamine monomer (d) of the polyimide (gIV) in the fifth embodiment of the present invention. The group IV is taken as the main chain of the dianhydride monomer (eIV) of the polyimide (gIV) in the fifth embodiment of the present invention. The formula of the polyimide (gIV) in the fifth embodiment of present invention is shown as formula (XIII) below:

[chemical structure]

It is worth noting that the formation of the polyimide (gIV) comprises thermal imidization to form polyimide (g′IV) and chemical imidization to form polyimide (g″IV). The method of thermal imidization and chemical imidization had already been described above and would be omitted thereafter.

### Sixth Embodiment

1,4-bis(4-aminophenoxy)-2-adamantyl benzene is taken as the diamine monomer (d) of the polyimide (gV) in the sixth embodiment of the present invention. The group V is taken as the main chain of the dianhydride monomer (eV) of the polyimide (gV) in the sixth embodiment of the present invention. The formula of the polyimide (gV) in the sixth embodiment of present invention is shown as formula (XIV) below:

[chemical structure]

It is worth noting that the formation of the polyimide (gV) comprises thermal imidization to form polyimide (g′V) and chemical imidization to form polyimide (g″V). The method of thermal imidization and chemical imidization had already been described above and would be omitted thereafter.

### Seventh Embodiment

1,4-bis(4-aminophenoxy)-2-adamantyl benzene is taken as the diamine monomer (d) of the polyimide (gVI) in the seventh embodiment of the present invention. The group VI is taken as the main chain of the dianhydride monomer (eVI) of the polyimide (gVI) in the seventh embodiment of the present invention. The formula of the polyimide (gVI) in the seventh embodiment of present invention is shown as formula (XV) below:

[chemical structure]

It is worth noting that the formation of the polyimide (gVI) comprises thermal imidization to form polyimide (g′VI) and chemical imidization to form polyimide (g″VI). The method of thermal imidization and chemical imidization had already been described above and would be omitted thereafter.

### Eighth Embodiment

1,4-bis(4-aminophenoxy)-2-adamantyl benzene is taken as the diamine monomer (d) of the polyimide (gVII) in the eighth embodiment of the present invention. The group VII is taken as the main chain of the dianhydride monomer (eVII) of the polyimide (gVII) in the eighth embodiment of the present invention. The structure formula of the polyimide (gVII) in the eighth embodiment of present invention is shown as formula (XVI) below:

[chemical structure]

It is worth noting that the formation of the polyimide (gVII) comprises thermal imidization to form polyimide (g′VII) and chemical imidization to form polyimide (g″VII). The method of thermal imidization and chemical imidization had already been described above and would be omitted thereafter.

### Ninth Embodiment

1,4-bis(4-aminophenoxy)-2-adamantyl benzene is taken as the diamine monomer (d) of the polyimide (gVIII) in the ninth embodiment of the present invention. The group VIII is taken as the main chain of the dianhydride monomer (eVIII) of the polyimide (gVIII) in the ninth embodiment of the present invention. The formula of the polyimide (gVIII) in the ninth embodiment of present invention is shown as formula (XVII) below:

[chemical structure]

It is worth noting that the formation of the polyimide (gVIII) comprises thermal imidization to form polyimide (g′VIII) and chemical imidization to form polyimide (g″VIII). The method of thermal imidization and chemical imidization had already describe detailed as above and would be omitted thereafter.

In the following paragraph, the analysis of the property of both polyimide compounds (g′) and polyimide compounds (g″) in the second to ninth embodiment are conducted. The property analysis comprises the viscosity test, the molecular weight test (such as the number average molecular weight Mn, average molecular weight Mw and polydispersity index Mw/Mn etc.), the solubility test, the mechanical property test (such as strength to break, elongation to break, and initial modulus etc.), and the thermal property test (such as glass transition temperature Tg, the thermal degradation temperature in nitrogen, and the thermal degradation temperature in oxygen). Table 1 is the result of the viscosity test and the molecular test of polyimide (g′I to g′VIII, g″I to g″VIII). Table 2 is the result of solubility test of polyimide (g′I to g′VIII, g″I to g″VIII). Table 3 is the result of the mechanical property of polyimide (g″I to g″VIII). Table 4 is the result of the thermal property test of polyimide (g″I to g″VIII).

TABLE 1

| | polyamic acid(f) | polyimide(g) | | |
|---|---|---|---|---|
| | Viscosity | | | Viscosity |
| | ηᵢₙₕ(dL/g) | Mn × 10−4 | Mw/Mn | ηᵢₙₕ(dL/g) |
| g′ I | 0.5 | — | — | — |
| g′ II | 0.42 | — | — | — |
| g′III | 1.98 | — | — | — |
| g′IV | — | — | — | — |
| g′V | 1.4 | — | — | — |
| g′VI | 1.15 | 9 | 1.81 | 1.11 |
| g′VII | 2.08 | — | — | — |
| g′VIII | — | — | — | — |
| g″ I | — | — | — | — |
| g″ II | — | 15.9 | 2.41 | 0.86 |
| g″III | — | 12 | 1.86 | 0.97 |
| g″IV | — | 16.6 | 1.92 | 1.54 |
| g″V | — | 11.5 | 1.89 | 0.93 |
| g″VI | — | — | — | — |
| g″VII | — | 14.2 | 1.98 | — |
| g″VIII | — | 4.8 | 1.41 | 0.67 |

Please refer to the results shown in Table 1. The viscosity of polyamic acid (f′I to f′VIII) is in the range of 0.40 to 2.08 dL/g. The viscosity of solvable polyimide (g′I to g′VIII) is in the range of 0.67 to 1.54 dL/g. The Mn measured by the GPC is in the range of 48,000 to 166,000.

TABLE 2

| | | | o- | m- | | |
|---|---|---|---|---|---|---|
| | NMP | DMAc | Chlorophenol | Cresol | Chloroform | THF |
| g′ I | − | − | − | − | − | − |
| g′ II | − | − | − | − | − | − |
| g′ III | − | − | +− | +− | +− | − |
| g′ IV | / | / | / | / | / | / |
| g′ V | − | − | +− | +− | − | − |
| g′ VI | +− | +− | +− | +− | + | − |
| g′ VII | − | − | +− | +− | +− | − |
| g′ VIII | ++ | ++ | + | + | +− | +− |
| g″ I | +− | − | − | − | − | − |
| g″ II | + | +− | ++ | + | +− | − |
| g″ III | + | + | + | + | ++ | +− |
| g″ IV | ++ | ++ | +− | +− | +− | +− |
| g″ V | ++ | ++ | + | +− | +− | +− |
| g″ VI | / | / | / | / | / | / |
| g″ VII | +− | +− | +− | +− | +− | − |
| g″ VIII | / | / | / | / | / | / |
| BAPB-IXb | − | − | − | − | − | − |
| BAPB-IXb | +− | +− | +− | +− | − | − |

In the solubility test, N-methyl-2-pyrrolidone, N,N-dimethylacetamide (DMAc), o-chlorophenol, m-cresol, chloroform, and tetrahydrofuran are utilized to test the solubility of polyimide (gI to gVIII). The result of the solubility of the solubility is shown in Table 2. It is worth noting that, in Table 2, “++” refers to soluble at room temperature, “+” refers to soluble at 60° C., “+−” refers to partial soluble at 60° C., and “−” refers to insoluble at 60° C.

As the result shown in the table 2, the solubility of polyimide compound (g″) made by chemical imidization is higher than which made by thermal imidization. The reason bringing to the poor solubility of the polyimide compound (g′) might be the crosslinking effect shown in part of polyamic acid (f′) in the process of the thermal imidization. The crosslinking effect would result in the greater intermolecular force of polyimide compound (g′), thus the solubility of polyimide compound (g′) would decrease. In addition, because of the poor solubility of polyimide (g′), the mechanical test and the thermal test of polyimide (g′) is omitted thereafter.

On the other hand, 1,4-bis(4-aminophenoxy)benzene (BAPB) and 1,4-bis(4-aminophenoxy)-2-phenyl benzene (BAPPB) are utilized as diamine monomer (d) in the present invention. The above-mentioned diamine monomer (d) and dianhydride monomer (eII) are utilized to form polyimide (BAPB-II, BAPPB-II), so as to make the solubility test as the control. As the result shown in Table 2, the solubility of polyimide compound (g″), having adamantyl as a functional group, is enhanced in contrast with the solubility of the control.

Besides, the main reason bringing to the better solubility of polyimide (g″IV) in all kinds of solvent might be the group “—CF3” inside the polyimide (g″IV). The F atom has large group, thus the arrangement between the molecules would be loose, and the solvent can be diffused into the molecular much easier. The solubility of polyimide (g″IV) would be enhanced. Furthermore, the solubility of polyimide (g″II, g″III, g″V, g″VI, g″VIII) are also soluble in several solvents.

According to the above-mentioned compared result, the conclusion can be made that the larger the size of the side chain introduced to the polyimide, the higher the solubility of the polyimide.

TABLE 3

| | Strength | Elongation | Initial |
|---|---|---|---|
| | to Break (MPa) | to Break (%) | Modulus (GPa) |
| g″ I | 91.3 | 4.2 | 2.3 |
| g″ II | 99.6 | 3.4 | 2.3 |
| g″III | 107.9 | 6.5 | 2.2 |
| g″IV | 95.9 | 3.8 | 2.3 |
| g″V | 90.9 | 4.4 | 2.1 |
| g″VI | 95.1 | 8.8 | 2.1 |
| g″VII | 125.1 | 11.1 | 2.3 |
| g″VIII | 93.7 | 1.2 | 2.3 |

Table 3 is the result of mechanical test of polyimide (g″I to g″VIII). As the result shown in Table 3, the strength to break polyimide (g″I to g″VIII) is in the range of 90.9 to 125.1 MPa, the value of elongation to break is in the range of 1.2 to 11.1%, and the initial modulus is in the range of 2.1 to 2.3 GPa.

TABLE 4

| | DSC, Tg | Decomposition in N2 | Decomposition in Air |
|---|---|---|---|
| g″ I | 352 | 502 | 507 |
| g″ II | 304 | 514 | 512 |
| g″III | 283 | 511 | 518 |
| g″IV | 312 | 495 | 592 |
| g″V | 245 | 521 | 524 |
| g″VI | 260 | 518 | 521 |
| g″VII | 301 | 511 | 511 |
| g″VIII | 299 | 500 | 503 |

As the result shown in table 4, the polyimide (g″I to g″VIII) have great property of heat resistance. In the nitrogen, the main thermal degradation temperature is in the range of 495 to 521° C. In the air, the main thermal degradation temperature is in the range of 503 to 592° C. According to the above-mentioned result, the polyimide (g″I to g″VIII) with aliphatic have greater thermal property.

In addition, the analyzed result of DSC shows that the Tg of polyimide (g″I to g″VIII) is in the range of 245 to 352° C. The strong structure of aliphatic group would cause the higher Tg of the polymer. The Tg of polyimide (g″I to g″IV, g″VII and g″VIII) is higher than 280° C. The Tg of polyimide (g″V, g″VI) is lower than above-mentioned polyimide (g″I to g″IV, g″VII and g″VIII). The lower Tg of polyimide (g″V, g″VI) might be caused by the asymmetrical structure and several soft groups such as ether. The asymmetrical structure and several soft groups would decrease the intermolecular force and cause lower Tg. The Tg sequence of polyimide (g″I to g″VIII) from high to low is g″I, g″IV, g″II, g″VII, g″VIII, g″III, g″VI, g″V as shown in the table 4.

To sum up, the present invention provides diamine monomer, polyimide compound and the manufacturing method thereof. The above-mentioned diamine monomer has larger size of the side chain, and thus the polyimide compound made of this kind of diamine monomer would have interrupted symmetry in structure and regularity in molecular arrangement. In addition, according to the result of the solubility test, the polyimide compounds having larger size side chain s are soluble in various kinds of solvent. Furthermore, above-mentioned polyimide compounds can still maintain desired mechanical and thermal property.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A diamine monomer having side chain, comprising the formula is listed as formula (I) below [chemical structure]
wherein R comprises aromatic hydrocarbon or alicyclic hydrocarbon.

2. The diamine monomer having side chain of claim 1, wherein aromatic hydrocarbon comprises phenyl, naphthalyl, anthracenyl or phenanthrenyl, and alicyclic hydrocarbon comprises adamantyl or diamantyl.

3. The diamine monomer having side chain of claim 1, wherein the R is a-substitution group.

4. A manufacturing method of diamine monomer having side chain, comprising:
(A) heat refluxing the solution containing bromo compound (a), hydroquinone compound and benzene to separate out solid hydroquinone compound (b),
wherein the reaction temperature is 80-85° C., and the reaction time is 3-4 days,
wherein the formula of bromo compound (a) is shown as formula (II) below: [chemical structure]
wherein the general formula of hydroquinone compound (b) is shown as formula (III) below: [chemical structure]
(B) heating the solution containing hydroquinone compound (b), potassium carbonate, 4-nitrochlorobenzene and dimethylformamid, and precipitating out the bis(4-nitrophenoxy) compound (c) by the distilled water,
wherein the reaction temperature is 120 to 140° C., and the reaction time is 8 to 14 hours,
wherein the general formula of bis(4-nitrophenoxy) compound (c) is shown as formula (IV) below: [chemical structure]
and
(C) heating refluxing the solution containing bis(4-nitrophenoxy) compound, hydrazine monohydrate, ethanol, and Pd/C catalyst to separate out the solid diamine monomer (d),
wherein the reaction temperature is 95 to 115° C., and the reaction time is 1 to 2 days,
wherein the general formula of diamine monomer (d) is shown as formula (I) below: [chemical structure]
wherein R group comprises aromatic hydrocarbon or alicyclic hydrocarbon.

5. The manufacturing method of diamine monomer having side chain of claim 4, wherein aromatic hydrocarbon comprises phenyl, naphthalyl, anthracenyl or phenanthrenyl, and alicyclic hydrocarbon comprises adamantyl or diamantyl.

6. The manufacturing method of diamine monomer having side chain of claim 4, wherein the R is α-substitution group.

7. The manufacturing method of diamine monomer having side chain of claim 4, wherein after the process of forming solid hydroquinone compound (b) further comprises:
washing solid hydroquinone compound (b) by the heated water,
wherein the temperature of the heated water is 70 to 80° C.; and
recrystallizing hydroquinone compound (b).

8. The manufacturing method of diamine monomer having side chain of claim 4, wherein the process after forming bis(4-nitrophenoxy) compound (c) further comprises:
washing bis(4-nitrophenoxy) compound (c) by the distilled water;
vacuum drying bis(4-nitrophenoxy) compound (c); and
recrystallizing bis(4-nitrophenoxy) compound (c) by the solvent comprising tetrahydrofuran, methanol, and water.

9. The manufacturing method of diamine monomer having side chain of claim 4, wherein after the process of forming diamine monomer (d) further comprises:
washing diamine monomer (d) by the heated distilled water, wherein the temperature of the heated water is 70 to 80° C.;
vacuum drying diamine monomer (d); and
recrystallizing diamine monomer (d) by the solvent comprises chloroform and n-hexane.

10. A polyimide compound having side chain, comprising the structure formula is shown as formula (IX) below: [chemical structure]
wherein R group comprises aromatic hydrocarbon or alicyclic hydrocarbon,
wherein R′ is selected from the group consisting of:

[chemical structure]

11. The polyimide compound having side chain of claim 10, wherein aromatic hydrocarbon comprises phenyl, naphthalyl, anthracenyl or phenanthrenyl, and the alicyclic hydrocarbon comprises adamantyl or diamantyl.

12. The polyimide compound having side chain of claim 10, wherein the R is α-substitution group.

13. A manufacturing method of polyimide compound having side chain, comprising:
(A) dissolving dianhydride monomer, diamine monomer into cresol by stirring under anhydrous condition to form polyamic acid compound solution,
wherein the general formula of diamine monomer is shown as formula (I) below: [chemical structure]
wherein the general formula of dianhydride monomer is shown as formula (VII) below: [chemical structure]
wherein the general formula of polyamic acid compound is shown as formula (VIII) below: [chemical structure]
wherein R comprises aromatic hydrocarbon or alicyclic hydrocarbon,
wherein R′ is selected from the group consisting of: [chemical structure]
wherein the reaction temperature is 20 to 30, the reaction time is 6 to 7 hours;
(B) adding 5-10 drops of isoquinoline to polyamic acid compound solution, and heating refluxing the solution to form polyimide compound solution,
wherein the reaction temperature is 100 to 200° C., the reaction time is 8 to 12 hours; and
(C) cooling polyimide compound solution and adding polyimide compound solution into ethanol to precipitate out solid polyimide compound,
wherein the general formula of polyimide compound is shown as formula (IX) below:

[chemical structure]

14. The manufacturing method of polyimide compound having side chain of claim 13, wherein aromatic hydrocarbon comprises phenyl, naphthalyl, anthracenyl or phenanthrenyl, and alicyclic hydrocarbon comprises adamantyl or diamantyl.

15. The manufacturing method of polyimide compound having side chain of claim 13, wherein the R is α-substitution group.

16. The manufacturing method of polyimide compound having side chain of claim 13, wherein cresol comprises p-cresol or m-cresol.

17. The manufacturing method of polyimide compound having side chain of claim 13, wherein after the process of forming the solid polyimide compound further comprises:
heating refluxing solid polyimide compound and ethanol,
wherein the reaction temperature is 70 to 80° C., the reaction time is 4 to 6 hours.

18. The manufacturing method of polyimide compound having side chain of claim 13, wherein the process of heating refluxing the solution comprising polyamic acid compound and isoquinoline further comprises:
heating refluxing the solution comprising polyamic acid compound and isoquinoline for 90-100° C.,
wherein the reaction time is 2 to 4 hours;
raising the reaction temperature to 140-150° C. and heating refluxing the solution comprising polyamic acid compound and isoquinoline,
wherein the reaction time is 2 to 4 hours; and
raising the reaction temperature to 200-210° C. and heating refluxing the solution comprising polyamic acid compound and isoquinoline,
wherein the reaction time is 2 to 4 hours.
